(19) United States
(12) Patent Application Publication
(10) Pub. No.: US 2013/0289572 A1
(43) Pub. Date: Oct. 31, 2013

(54) POSITIONAL GUIDE

(71) Applicant: Charles Brandon Broome, South Carolina, NY (US)

(72) Inventor: Charles Brandon Broome, South Carolina, NY (US)

(73) Assignee: KYPHON SARL, Neuchatel (CH)

(21) Appl. No.: 13/455,212

(22) Filed: Apr. 25, 2012

(51) Int. Cl. A61B 17/90; A61B 17/88

(52) U.S. Cl. 606/96

(57) ABSTRACT

A positional guide for use on a cannula shaft includes a shaft portion configured to form a first opening to receive a cannula shaft therein. One or more offset portions are connected to the shaft portion and configured to form second openings spaced apart from the first opening. The second openings are configured to each receive a stabilization device therein such that when the cannula shaft and the stabilization device are present in the first opening and the second openings, respectively, the cannula shaft and the stabilization device are disposed substantially parallel to each other. Methods are also disclosed.

### TECHNICAL FIELD

The present disclosure provides a positional guide to control spacing between a cannula and a stabilization wire or rod for use in surgical procedures and, more particularly, to a k-wire positional guide configured to permit uni-directional balloon inflation in, for example, the treatment of fractures. Methods of using such a device are also provided.

### BACKGROUND

During minimally invasive surgical procedures, cannulas are utilized to provide an access port for surgical instruments and for treating conditions such as fractures. A sharp trocar may be positioned within the cannula and utilized to puncture or pierce the tissue to access the damaged bone. Thereafter, the trocar may be removed, leaving the cannula in place providing the access port to the site of treatment. Stabilization of the cannula during treatment is very important, as the ports can be accidentally ejected from the patient or moved while materials, such as bone cement, are being delivered resulting in less perfect treatment and increased procedure time.

In a bone, where there is minimal cancelous bone, such as a distal radius, when the cannula is inserted, it cannot be easily stabilized and does not remain stationary. This destabilization can cause imprecise disbursement of material, such as bone cement. Balloons are also used in treating fractures and in spinal procedures for creating a void for injection of bone cement and other materials.

### SUMMARY

This application relates to positional guides and methods for use. In accordance with the present principles, a positional guide for use on a cannula shaft includes a shaft portion configured to form a first opening to receive a cannula shaft therein. One or more offset portions are connected to the shaft portion and configured to form second openings spaced apart from the first opening. The second openings are configured to each receive a stabilization device therein such that when the cannula shaft and the stabilization device are present in the first opening and the second openings, respectively, the cannula shaft and the stabilization device are disposed substantially parallel to each other.

In another embodiment, a positional guide for use on a cannula shaft includes a shaft portion configured to form an opening to receive a cannula shaft therein. A monolithic block has a plurality of holes formed therethrough for receiving wires or pins therein. The monolithic block is connected by an arm forming a hooked end portion, which forms the opening and is configured to laterally receive the cannula shaft mid-span. The plurality of holes is positioned relative to at least one base circle diameter such that a center of the base circle is coincident with a center of the opening to receive the cannula shaft.

A treatment method includes providing a positional guide having a shaft portion configured to form a first opening to receive a cannula shaft therein, one or more offset portions connected to an exterior of the shaft portion and configured to form second openings to receive one of a wire or pin therein; positioning the cannula shaft through the first opening of the positional guide; placing the wires or pins in the second openings of the positional guide such that the cannula shaft and the wires or pins are disposed substantially parallel to each other; securing the wires or pins to bone; and treating the bone.

### BRIEF DESCRIPTION OF THE DRAWINGS

The present disclosure will become more readily apparent from the specific description accompanied by the following drawings, in which:

FIG. 1 is a front view of a positional guide having openings for a cannula shaft and for stabilization devices (e.g., k-wires or pins) in accordance with the principles of the present disclosure;

FIG. 2 is a side view of the positional guide of FIG. 1 showing, in dashed lines, a cannula shaft and k-wires in one particular embodiment;

FIG. 3 is a perspective view of the positional guide of FIGS. 1 and 2 in use during a bone treatment procedure in accordance with one illustrative embodiment;

FIG. 4 is a front view of a positional guide showing a plurality of holes in a block portion configured on a base circle to provide a working relationship with an inflatable device in accordance with one embodiment;

FIG. 5 is a perspective view of the positional guide of FIG. 4 in use during a bone treatment procedure in accordance with another illustrative embodiment; and

FIG. 6 is a magnified view of the positional guide shown in FIG. 5 in accordance with the present principles.

Like reference numerals indicate similar parts throughout the figures.

### DETAILED DESCRIPTION

An apparatus and method are described for a device, system and method employed with a cannula for aligning and maintaining medical instruments. In one embodiment, a device or positional guide is provided that receives Kirschner wires (k-wires) or pins and engages a medical device such as a cannula. The guide includes one or more through holes or openings that receive the k-wires or pins. The guide is adapted to detachably receive the medical instrument being employed. In one case, the medical instrument includes a cannula and the guide connects or snaps onto a shaft portion of the cannula. In particularly useful embodiments, the holes in the guide are configured to permit the use of the k-wires or pins with a balloon or inflatable bone tamp (IBT). The holes are arranged so as to not interfere with the expansion of the balloon when the k-wires are inserted through the holes. In another embodiment, the holes are configured to provide interference with the balloon so that uni-directional motion of the balloon is achieved.

For illustrative purposes, the guide and method shall be described in the context of bone treatment, e.g., injecting bone void filler/bone cement into a portion of a long bone or vertebra of a patient to treat bone fractures, although the apparatus and methods can be used to treat other conditions.

Kirschner wires or k-wires are sterilizable, sharpened, smooth stainless steel pins widely used in orthopedics and other types of medical and veterinary surgery. K-wires are available in different sizes and are used to hold bone fragments together (pin fixation) or to provide an anchor for skeletal traction. The pins are often driven into the bone through the skin (percutaneous pin fixation) using a power or hand drill. The guide and method will illustratively be described in terms of k-wires and pins although other stabilization devices may be employed.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, distal and proximal, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament and/or bone, repairing a fracture or break in bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

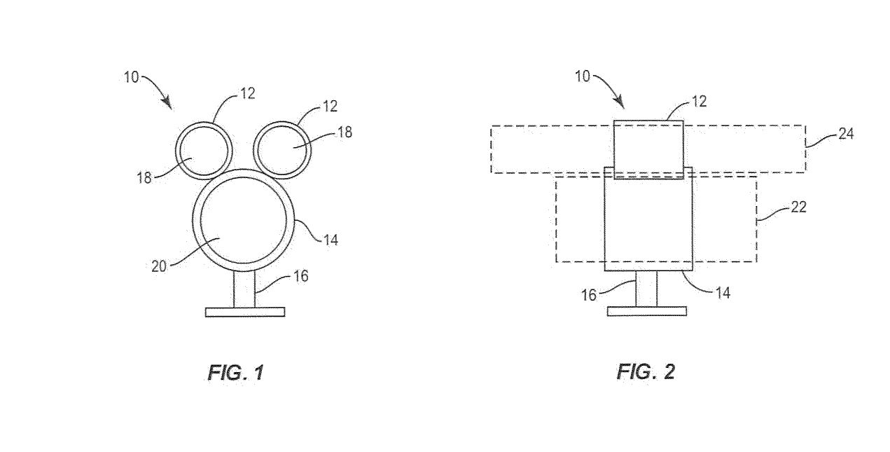

The following disclosure includes a description of a position guide and system that can be employed with a cannula for treating fractures, including using an inflatable bone tamp, using drills and delivering bone cement/void filler, such as, for example, auto graft, allograft, demineralized bone material, mineral composites, blocks, granules and pellets and bone cement, such as, for example, PMMA-based material (HVR, Activos, Activos 10, Spine-05), Calcium Phosphate (Skaffold, Norian, Hydroset, Kyphos FS) and Calsium Sufacte (Wright Medical), as well as other injectables. The disclosure also includes a description of related methods of employing the guide with a cannula and an inflatable bone tamp in accordance with the present principles. Alternate embodiments are also disclosed. Reference will now be made in detail to the exemplary embodiments of the present disclosure, which are illustrated in the accompanying figures. Turning now to FIGS. 1-6, there are illustrated components of a position guide 10 in accordance with the principles of the present disclosure.

The components of the guide 10 can be fabricated from biologically acceptable materials suitable for medical apparatuses, including metals, synthetic polymers, ceramics, thermoplastic and polymeric material and/or their composites. For example, the components of the guide 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan, Fe—Mn—Si and Fe—Ni—Co—Ti composites), ceramics and composites thereof such as calcium phosphate (e.g., SKELITE™ manufactured by Biologix™, Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers based materials, polymeric rubbers, polyolefin rubbers, semi-rigid and rigid materials, thermoplastic elastomers, thermoset elastomers, elastomeric composites, rigid polymers including polyphenylene, polyamide, polyimide, polyetherimide, polyethylene, epoxy, polyacrylate and composites of metals and calcium-based ceramics, composites of PEEK and calcium based ceramics, and combinations of the above materials. Various components of the guide 10 may have material composites, including the above materials, to achieve various desired characteristics such as strength, rigidity, elasticity, compliance, and biomechanical performance, durability and to provide a non-stick surface. The components of the guide 10 may be monolithically formed, integrally connected or include fastening elements and/or coupling components, as described herein. The guide 10 as described herein may be disposable or may be autoclavable/resterilizable. The guide 10 may be part of a kit.

In one embodiment, as shown in FIG. 1, the guide 10 is depicted in a simple form. The guide 10 includes a main body 14 and two offset bodies 12 rigidly attached to the main body 14. It should be noted that the guide 10 may include as few as one offset body 12 or as many offset bodies 12 as needed or desired. In an alternate embodiment, the guide 10 includes a monolithic structure having a plurality of holes formed therein. The main body 14 forms an opening 20, which is configured to receive a cannula or other instrument during a procedure. Offset bodies 12 may also be configured to receive a stabilization device, such as, e.g., k-wires or pins. The k-wires may be employed to stabilize the cannula and may be provided in a parallel orientation with respect to the cannula. An optional fastening device or locking mechanism 16 may be included. The fastening device 16 may include a screw, a protrusion, a clamping mechanism or other device that can be advanced, clamped or closed to grip a shaft portion 22 (FIG. 2) of the cannula to prevent the guide 10 from sliding down a cannula shaft. Alternately, the mechanism 16 may not be employed, and the guide is slidably movable along a longitudinal axis of the cannula shaft and/or the stabilization device or devices.

Referring to FIG. 2, a side view of the positional guide 10 is shown in accordance with the present principles. The guide 10 illustratively includes a portion of a k-wire 24 and a portion of a shaft 22 of a cannula to demonstrate the use of the guide 10. In this embodiment, the cannula shaft 22 is positioned in the main body 14 of the guide 10. The k-wire or k-wires 24 are passes through the offset bodies 12. The k-wires 24 are employed to stabilize and align the cannula during a procedure. In accordance with the present principles, the offset bodies 12 may be configured and dimensioned so as not to interfere with an inflatable element (e.g., a balloon or inflatable bone tamp (IBF)) employed during a procedure.

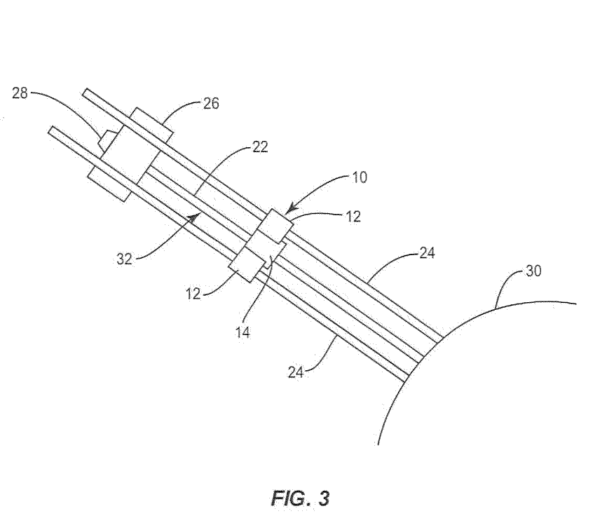

Referring to FIG. 3, an illustrative configuration is depicted showing a positional guide 10 mounted on a cannula 32 and guiding k-wires 24 therethrough. The cannula 32 includes a handle 26 having a proximal port 28. The port 28 communicates with a lumen in the shaft 22 and permits instruments or materials (e.g., bone cement) to pass through the lumen into a body 30 of a subject. The guide 10 permits the shaft 22 and the k-wires 24 to be deployed in a parallel orientation and to maintain a relative distance between the shaft 22 and the k-wires 24.

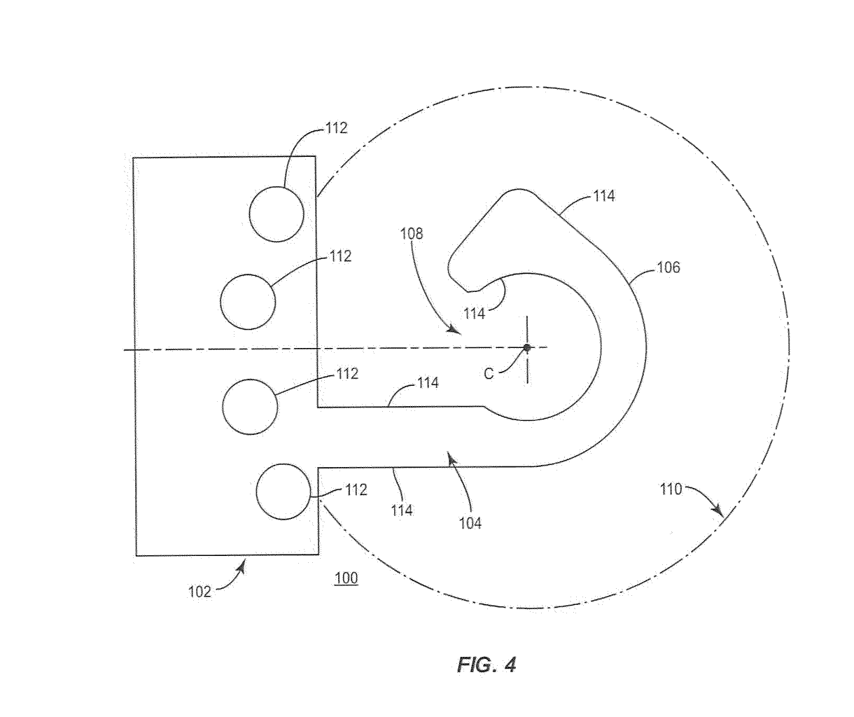

Referring to FIG. 4, a positional guide 100 is shown in accordance with another embodiment. Guide 100 includes a block portion 102, and an arm portion 104, which extends from the block portion 102. The arm portion 104 forms a circular extension 106 that forms a cavity or opening 108 for receiving a medical instrument (not shown) such as a shaft of a cannula. The guide 100 includes chamfers 114 (or radii) configured to ease usage, reduce stress risers and provide better fits for the equipment that interfaces with the guide 100. The block portion 102 includes a plurality of openings or holes 112 formed therein. The holes 112 are configured to receive k-wires or pins.

The guide 100 is configured to accommodate a balloon or other expandable instrument (e.g., IBT) (not shown) at its fully extended position (e.g., at maximum inflation volume (MIV)) by spacing apart the holes 112 from a center C of a base circle 110. The base circle 110 has a coincident center with the cavity 108. The base circle 110 represents the MIV of a spherical balloon or IBT. The base circle 110 defines an inner boundary of the holes 112 such that k-wires passing through the holes 112 will not interfere with the expansion of the balloon. In another embodiment, the holes 112 may be located within the base circle 110 such that upon inflation of the balloon, a uni-directional displacement between the cannula shaft passing through cavity 108 and the k-wires through the holes 112 occurs. This may be employed when a uni-directional balloon expansion is needed.

The components of the guide 100 can be fabricated from biologically acceptable materials suitable for medical apparatuses, including metals, synthetic polymers, ceramics, thermoplastic and polymeric material and/or their composites as described above. The components of the guide 100 may be monolithically formed, integrally connected or include fastening elements and/or coupling components, as described herein. The guide 100 as described herein may be disposable or may be autoclavable/resterilizable. The guide 100 may be part of a kit. The arm portion 104 with the circular extension 106 may include an elastically deformable material. While the chamfer 114 in the cavity 108 may be employed to assist in passing the shaft of the cannula directly through the cavity 108, the circular extension 106 may be flexible to permit the cavity to be opened up so that the shaft can be inserted laterally mid-span on the shaft. An interference fit may be designed into the circular extension 106 so that a gripping of the cannula shaft can be achieved. Alternately, a clearance fit may be employed to permit the guide 100 to slide along the cannula shaft.

Other configurations of the guide 100 may be employed in accordance with the present principles. For example, a greater number of holes 112 may be employed, and the holes 112 may be located on different sized base circles (110). In another embodiment, gripping devices may be implemented for the holes 112 so that the stabilization devices passing through holes 112 may be gripped to prevent motion.

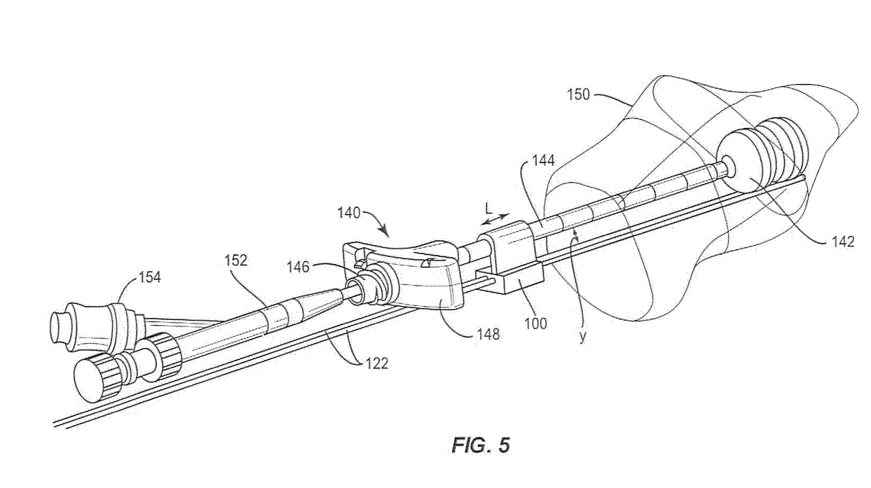

Referring to FIG. 5, the guide 100 is shown in use during a bone fracture repair procedure. A cannula device 140 includes a hub or handle 148 connected to a shaft 144. A port 146 is formed in the hub 148 and communicates with a lumen in the shaft 144. In one embodiment, bone cement or other materials may be injected into the port 146 using a syringe, pump or other device 152. An inflation device 154 may be employed to inflate a balloon or IBT 142. The guide 100 is deployed on the shaft 144 of the cannula device 140. K-wires 122 pass through the guide 100 in a substantially parallel configuration relative to the shaft 144. The shaft 144 and the k-wires 122 are held in a close, well defined relationship, by the guide 100. The balloon or IBT 142 is employed and connected to a distal end portion of the shaft 144. The balloon 142, the shaft 144 and the k-wires 122 are inserted into tissue 150, such as a bone and/or soft tissue.

During a procedure, the guide 100 is engaged on the shaft 144 of the cannula device 140 (this may be performed before or after inserting the cannula shaft 144 into tissue 150). The shaft 144 is inserted into the target tissue 150. K-wires 122 are threaded through the guide 100 and affixed to bone 150. By employing the guide 100 more accurate placement of the k-wires is enabled. In addition, the use of multiple k-wires 122 through the guide 100 increases the stability of the cannula and the repeatability of the positioning of the cannula shaft 144 during the procedure.

A distance y between the k-wires and an outer diameter of the balloon 142 may be selected in the design of the guide 100 to provide clearance with the balloon 142 or to provide a uni-directional movement of the balloon 142. As described above, if the k-wire guide holes 112 (FIG. 4) fall outside the base circle (110) corresponding to the balloon's maximum diameter, then the balloon 142 will not engage the k-wires 122. If, however, it is desirable to have the balloon 142 create a uni-directional motion of the shaft 144 from the k-wires 122, then the holes 112 should be disposed within the base circle 110 a predetermined amount to obtain the desired motion. As an example, the predetermined amount may be about 4 mm or so. As the balloon 142 is inflated, in the latter case, the dimension y increases to provide the uni-directional motion. Moving the guide 100 longitudinally along the cannula shaft 144 (in a direction L) can affect the amount of uni-directional motion in the y-direction since angle formed between the cannula shaft 144 and the k-wires 122 can change based on the longitudinal position of the guide 100.

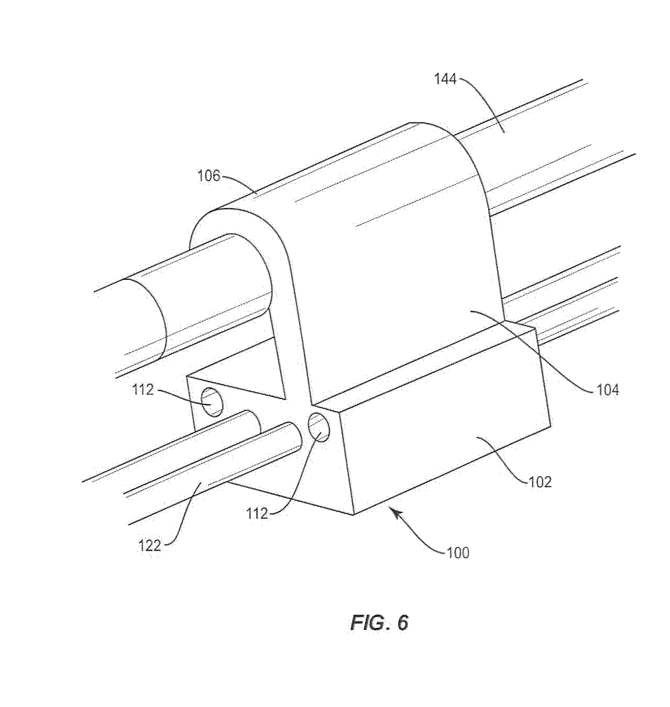

Referring to FIG. 6, the guide 100 of FIG. 5 is shown in a magnified view. While the guide 110 may include holes 112 on a same base circle, the holes 112 may be varied in dimension and in position on the body portion 102 such that use of different hole combinations for k-wires 122 may provide for full expansion of a balloon while a different set of holes may provide for overlap between the balloon expansion and the positions of k-wires 122 to provide the uni-directional motion of the balloon inflation. By providing a plurality of holes 112 on different base circles, a single guide 100 may be employed for different procedures or may be employed for different steps or functions in a same procedure.

The cannulas depicted in FIGS. 1-6 may take many forms. It is contemplated that the guide 10 or guide 100 may be variously configured and include different shaped portions, such as, for example, round, oval, oblong, square, rectangular, polygonal, irregular, uniform, non-uniform, offset, staggered, tapered, consistent or variable, depending on the requirements of a particular application. An outer surface of the guide may be rough, arcuate, undulating, mesh, porous, semi-porous, dimpled and/or textured according to the requirements of a particular application

Arm portions 104 of the guide 100 can be variously configured, such as, for example, round, oval, oblong, square, rectangular, polygonal, irregular, uniform, non-uniform, offset, staggered, tapered, consistent or variable, depending on the requirements of a particular application.

In assembly and use, guides may be employed during a surgical procedure for treatment of a disorder affecting a section of bone, such as a fracture, for example in the distal radius, tibial plateau or proximal humerus of a patient, as discussed herein. In use, to treat the affected section, a medical practitioner obtains access to the bone in any appropriate manner, such as through incision and retraction of tissues. Once the bone is exposed the cannula can be used to create and enter an access port adjacent to a fracture to be treated. It is envisioned that cannula may be used in any existing surgical method or technique including open surgery, mini-open surgery, minimally invasive surgery and percutaneous surgical implantation, whereby the site is accessed through a micro-incision, or sleeve that provides a protected passageway to the area. Once access to the surgical site is obtained, the cannula can be deployed so to deliver an agent, such as bone void filler/bone cement, to treat the condition, such as repair the fracture.

In one particular embodiment, the guide is applied on the cannula. The cannula is delivered into the surgical site adjacent to a fracture or a break in a portion of a bone such as the distal radius, tibial plateau or proximal humerus as well as other bone sites. These bone sites have minimal cancelous bone, and therefore, when the cannula is inserted, it cannot be easily stabilized and does not remain stationary. This unstabilization can cause imprecise disbursement of material, such as bone void filler/bone cement. To facilitate stabilization of the cannula, a balloon may be employed and when deployed is disposed between an expanded configuration and an unexpanded configuration. When the cannula is inserted into the surgical site, the configuration is in its unexpanded configuration within the cannula. As the balloon exits a distal end of the cannula, the expanded configuration is achieved by filling the balloon. The expanded configuration stabilizes the cannula in place by pressing against the existing cancelous bone thereby allowing for precise delivery of bone void filler/bone cement, or deployment of other agents and/or surgical tools that may be required. For further stability, k-wires are employed through the guide to further stabilize the cannula. The balloon is deflated to retract it back into the cannula, and the cannula system can be removed. The cannula shaft may pass directly through the balloon.

It is contemplated that in addition to bone void filler/bone cement, other agents can be delivered to a surgical site using the cannula. These agents include therapeutic polynucleotides or polypeptides. It is further contemplated that these agents may also include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines. The components of the guide can be made to include radiolucent materials such as polymers. Radiomarkers may be included for identification under x-ray, fluoroscopy, CT or other imaging techniques. It is envisioned that the agent may include one or a plurality of therapeutic agents and/or pharmacological agents for release, including sustained release, to treat, for example, pain, inflammation and degeneration.

It is envisioned that the use of microsurgical and image guided technologies may be employed to access, view and repair bone deterioration or damage, in conjunction with the cannula and positional guides.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A positional guide for use on a cannula shaft, comprising:
a shaft portion configured to form a first opening to receive a cannula shaft therein;
one or more offset portions connected to the shaft portion and configured to form second openings spaced apart from the first opening, the second openings configured to each receive a stabilization device therein such that when the cannula shaft and the stabilization device are present in the first opening and the second openings, respectively, the cannula shaft and the stabilization device are disposed substantially parallel to each other.

2. A positional guide of claim 1, wherein the first opening and the second openings form closed holes.

3. A positional guide of claim 1, wherein the first opening is open to permit lateral insertion mid-span of the cannula shaft.

4. A positional guide of claim 1, wherein the second openings are provided at a distance from the first opening to prevent interference with an expandable element at a distal end portion of the cannula shaft.

5. A positional guide of claim 1, wherein the second openings are provided at a distance from the first opening to provide interference with an expandable element at a distal end portion of the cannula shaft to permit a uni-directional movement due to an expansion of the expandable element.

6. A positional guide of claim 1, further comprising a fastening device associated with the first opening to prevent motion of the guide with respect to the cannula shaft.

7. A positional guide of claim 1, wherein the offset portion includes a monolithic block having a plurality of holes formed therethrough for receiving wires or pins therein as stabilization devices.

8. A positional guide of claim 7, wherein the plurality of holes are positioned relative to at least one base circle diameter, a center of the base circle being coincident with a center of the first opening.

9. A positional guide of claim 7, wherein the plurality of holes are positioned relative to at least two base circles having different diameters, centers of the base circles being coincident with a center of the first opening.

10. A positional guide of claim 1, wherein the first opening receives a cannula shaft and the second openings each receive a k-wire, each k-wire being secured to a bone, and further comprising an inflatable bone tamp associated with the cannula shaft for treatment of the bone.

11. A positional guide of claim 10, wherein at least two k-wires are disposed substantially parallel to each other through the positional guide.

12. A positional guide of claim 10, wherein the positional guide is slidable along the cannula shaft and the k-wires.

13. A positional guide of claim 1, wherein the one or more offset portions are connected to the shaft portion by an arm forming a hooked end portion, which forms the first opening, the first opening being open to laterally receive the cannula shaft mid-span.

14. A positional guide for use on a cannula shaft, comprising:
a shaft portion configured to form an opening to receive a cannula shaft therein;
a monolithic block having a plurality of holes formed therethrough for receiving wires or pins therein, the monolithic block being connected by an arm forming a hooked end portion, which forms the opening and is configured to laterally receive the cannula shaft mid-span, wherein the plurality of holes are positioned relative to at least one base circle diameter such that a center of the base circle is coincident with a center of the opening to receive the cannula shaft.

15. A positional guide of claim 14, wherein the plurality of holes are provided at a distance from the opening to prevent interference with an expandable element at a distal end portion of the cannula shaft.

16. A positional guide of claim 14, wherein the plurality of holes are provided at a distance from the opening to provide interference with an expandable element at a distal end portion of the cannula shaft to permit a uni-directional movement due to an expansion of the expandable element.

17. A positional guide of claim 14, wherein the plurality of holes are positioned relative to at least two base circles having different diameters and centers of the base circles being coincident with a center of the opening.

18. A treatment method, comprising:
providing a positional guide having a shaft portion configured to form a first opening to receive a cannula shaft therein, one or more offset portions connected to an exterior of the shaft portion and configured to form second openings to receive one of a wire or pin therein;
positioning the cannula shaft through the first opening of the positional guide;
placing the wires or pins in the second openings of the positional guide such that the cannula shaft and the wires or pins are disposed substantially parallel to each other;
securing the wires or pins to bone; and
treating the bone.

19. A method as in claim 18, wherein the step of placing the wires or pins in the second openings of the positional guide further comprises placing a plurality of wires or pins in the second openings which are positioned at a distance from the first opening to prevent interference with an expandable element employed at a distal end portion of the cannula shaft.

20. A method as in claim 18, wherein the step of placing the wires or pins in the second openings of the positional guide further comprises placing a plurality of wires or pins in the second openings which are provided at a distance from the first opening to provide interference with an expandable element at a distal end portion of the cannula shaft to permit a uni-directional movement due to an expansion of the expandable element.
